(19) United States
(12) Patent Application Publication
(10) Pub. No.: US 2025/0149157 A1
(43) Pub. Date: May 8, 2025

(54) EXAMINATION SUPPORT APPARATUS, MEDICAL EXAMINATION APPARATUS, EXAMINATION SUPPORT METHOD, AND EXAMINATION SUPPORT PROGRAM

(71) Applicant: FUJIFILM Corporation, Tokyo (JP)

(72) Inventor: Taiga GOTO, Tokyo (JP)

(73) Assignee: FUJIFILM Corporation, Tokyo (JP)

(21) Appl. No.: 18/936,944

(22) Filed: Nov. 4, 2024

(30) Foreign Application Priority Data
Nov. 6, 2023 (JP) 2023-189593

(51) Int. Cl. G16H 40/63; G06F 3/14; G06V 20/50; G06V 40/10; G10L 15/00

(52) U.S. Cl. CPC G16H 40/63; G06F 3/1423; G06V 20/50; G06V 40/10; G10L 15/005; G06V 2201/03

(57) ABSTRACT

An examination support apparatus includes a processor, in which the processor acquires target subject data including at least one of imaging data obtained by imaging a target subject who is a presentation destination of information related to a medical examination or audio data obtained by collecting voice of the target subject, and decides presentation information to be presented for the target subject by a presentation device that presents the information related to the medical examination, on the basis of the target subject data.

### CROSS-REFERENCE TO RELATED APPLICATIONS

The present application claims priority under 35 U.S.C. § 119 to Japanese Patent Application No. 2023-189593 filed on Nov. 6, 2023. The above application is hereby expressly incorporated by reference, in its entirety, into the present application.

### BACKGROUND

### Technical Field

The technology of the present disclosure relates to an examination support apparatus, a medical examination apparatus, an examination support method, and an examination support program.

### Related Art

JP2011-022740A discloses “a voice guidance management server and medical image diagnostic apparatus including: an examination/voice guidance correspondence storage unit that stores in advance an examination method and voice guidance corresponding to the examination method for each of a plurality of languages; an examination information acquisition unit that acquires examination information including an examination method of a patient registered in a reservation examination server and language information of the patent; and an examination information controller that acquires the examination method of the patient and the language information of the patient from the examination information acquisition unit, acquires the voice guidance corresponding to the examination method and the language of the patient from the examination/voice guidance correspondence storage unit, and notifies a medical image diagnostic apparatus of the voice guidance as examination/voice guidance information”.

JP2018-094209A discloses “an examination support system for a subject in an X-ray examination, which gives an instruction on an examination body position requested by an examiner to the subject and gives guidance regarding an explanation content related to the examination, between the examiner and the subject separated by a shield, the examination support system including an examination instruction operation terminal that includes an instruction information transmission unit having an operation instruction screen in which an instruction button is arranged for selecting instruction information on a body position instruction and an explanation content related to the examination and transmitting the instruction information using the instruction button selected and operated; and an examination instruction display terminal that includes an instruction information reception unit receiving the instruction information transmitted from the instruction information transmission unit and having an instruction display screen for displaying the body position instruction and the explanation content related to the examination in the instruction information.

JP5816192B discloses “a processing method executed by an image forming system including a display, the processing method including a step of displaying a description of an examination procedure on the display, and a step of scanning an examination region to form an image of a patient by an image forming apparatus, in which the processing method includes a step of displaying a question item to the patient via an output region of the display and receiving an answer to the question item from the patient via an input region of the display, at both time points before and after the step of forming the image”.

It is desired to make information regarding a medical examination into a format according to a person who is a presentation destination. Here, JP2011-022740A discloses that language information of a registered patient is acquired from a reservation examination server, and voice guidance is made to correspond to the language of the patient. In addition, JP2018-094209A discloses that a foreign language menu is displayed in a pull-down format and in a case where a predetermined foreign language is selected, an instruction to a subject is displayed in the selected foreign language. In addition, JP5816192B discloses that, in a case where a patient is a child, an animation or a comic of a radiologist and/or another child is displayed on a display as an assistant. Here, the age of the patient is acquired from information registered in advance in an electronic schedule or the like.

As described above, in the related art, it is necessary to register information regarding the subject in advance (including information to be registered immediately before information presentation). However, in the medical field, various kinds of information are registered in advance as the information regarding the subject. In addition, in a case where the subject is in a critical condition, a situation may occur in which information regarding the subject is not registered in advance and cannot be registered immediately before the information presentation due to injury or the like. Furthermore, the information regarding an assistant accompanying the subject is not registered in advance in practice. Therefore, there is a case where it is not possible to make information related to a medical examination into a format according to the subject, assistant, or the like as the presentation destination.

### SUMMARY

Therefore, an object of the technology of the present disclosure is to provide an apparatus, a method, and a program which can make information related to a medical examination into a format according to a person who is a presentation destination even in a case where the information regarding a person who is a presentation destination of the information is not registered in advance or is insufficiently registered.

In order to achieve the object, an examination support apparatus according to an aspect of the technology of the present disclosure includes a processor, and the processor acquires target subject data including at least one of imaging data obtained by imaging a target subject who is a presentation destination of information related to a medical examination or audio data obtained by collecting voice of the target subject, and decides presentation information to be presented for the target subject by a presentation device that presents the information related to the medical examination, on the basis of the target subject data.

The processor may recognize attributes of the target subject by using the target subject data, and decide the presentation information on the basis of the attributes.

In a case where a plurality of target subjects having different attributes are recognized, the processor may receive a user's input for selecting which target subject among the plurality of target subjects the presentation information is presented for.

In a case where a plurality of target subjects having different attributes are recognized, the processor may automatically select which target subject among the plurality of target subjects the presentation information is presented for, according to a standard determined in advance.

In a case where the presentation device includes a first presentation device and a second presentation device that present the information related to the medical examination in different formats and a first target subject and a second target subject having different attributes are recognized, the processor may decide each of first presentation information to be presented for the first target subject by the first presentation device and second presentation information to be presented for the second target subject by the second presentation device.

In a case where the attributes of the target subject include at least a language used by the target subject and the language used cannot be uniquely recognized, the processor may decide the presentation information such that the presentation device presents the information related to the medical examination for the target subject in a plurality of candidate languages.

The processor may decide the presentation information such that the presentation device presents the information related to the medical examination for the target subject in a format according to a recognition accuracy for each of the plurality of candidate languages.

The processor may recognize a situation of the target subject during viewing of the information related to the medical examination by using the target subject data, and decide the presentation information on the basis of the situation of the target subject.

The processor may acquire registration information registered in advance for the target subject, and decide the presentation information on the basis of the registration information and the target subject data.

In a case where the presentation information cannot be decided on the basis of the registration information, the processor may instruct an imaging device, which images the target subject, to perform imaging or instruct a sound collecting device, which collects voice of the target subject, to collect sound.

In a case where the presentation device includes an acoustic device that presents the information related to the medical examination by voice, the processor may decide at least any of a frequency, a volume, a speed, or a language of the voice in deciding the presentation information.

In a case where the presentation device includes a display device that presents the information related to the medical examination by display, the processor may decide at least any of a color, a brightness, a size, a speed, or a language of the display in deciding the presentation information.

A medical examination apparatus according to another aspect of the technology of the present disclosure includes the examination support apparatus.

An examination support method according to another aspect of the technology of the present disclosure includes, via a computer, acquiring target subject data including at least one of imaging data obtained by imaging a target subject who is a presentation destination of information related to a medical examination or audio data obtained by collecting voice of the target subject; and deciding presentation information to be presented for the target subject by a presentation device that presents the information related to the medical examination, on the basis of the target subject data.

An examination support program according to another aspect of the technology of the present disclosure causes a computer to execute acquiring target subject data including at least one of imaging data obtained by imaging a target subject who is a presentation destination of information related to a medical examination or audio data obtained by collecting voice of the target subject; and deciding presentation information to be presented for the target subject by a presentation device that presents the information related to the medical examination, on the basis of the target subject data.

According to the technology of the present disclosure, even in a case where a person who is a presentation destination of information is not registered in advance or is insufficiently registered, it is possible to make information related to a medical examination into a format according to the person who is the presentation destination.

### BRIEF DESCRIPTION OF THE DRAWINGS

FIG. 1 is a diagram illustrating an example of a schematic configuration of a medical examination system 1 according to the present embodiment.

FIG. 2 is a diagram illustrating an example of a hardware configuration of an examination support apparatus 100 according to the present embodiment.

FIG. 3 is a diagram illustrating an example of a functional configuration of the examination support apparatus 100 according to the present embodiment.

FIG. 4 is a diagram illustrating an example of a flow in a case where information related to a medical examination is presented for a subject 10.

FIG. 5 is a diagram illustrating an example of a flow in a case where information related to a medical examination is presented for an assistant 30.

FIG. 6 is a diagram illustrating an example of display in a case where a user's input is received.

FIG. 7 is a diagram illustrating an example of a display in a case where presentation information is presented in a plurality of candidate languages.

### DETAILED DESCRIPTION

Hereinafter, examples of embodiments of the present disclosure will be described with reference to the drawings. Note that, in each drawing, the same or equivalent constituent elements and parts are given the same reference numerals. In addition, dimensional ratios in the drawings are exaggerated for convenience of description and may be different from the actual ratios.

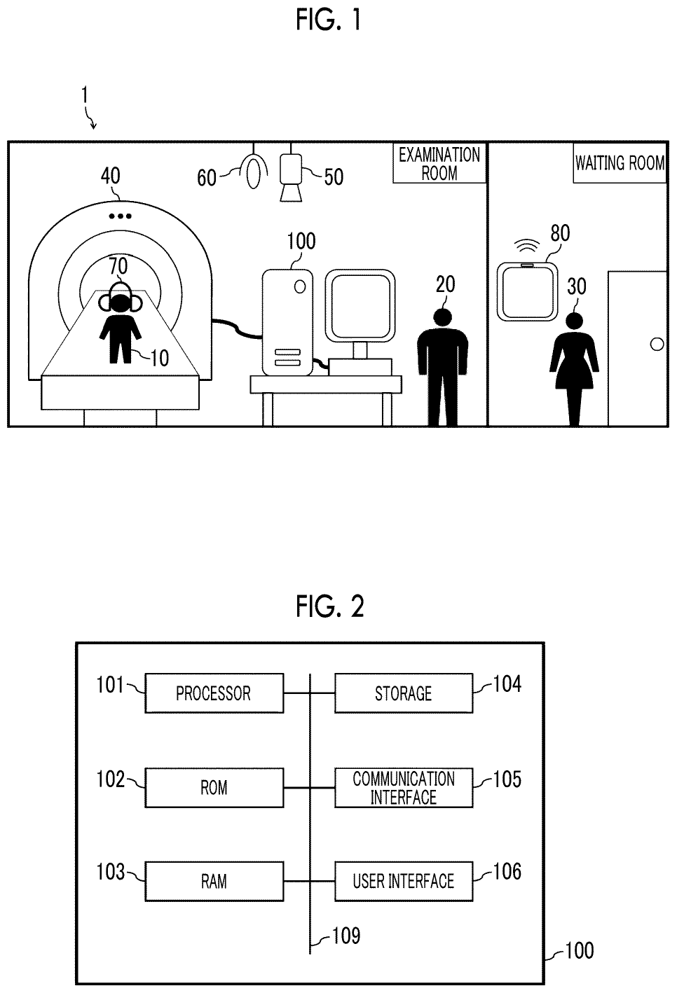

FIG. 1 is a diagram illustrating an example of a schematic configuration of a medical examination system 1 according to the present embodiment. In FIG. 1, as an example, a case where the medical examination system 1 is a system related to a magnetic resonance imaging (MRI) examination. However, the present disclosure is not limited to this configuration. The medical examination system 1 may be a system related to any medical examination such as computed tomography (CT), positron emission tomography (PET), mammography, general X-rays, ultrasound waves, or an endoscope.

In FIG. 1, as an example, a state is illustrated in which a subject 10 receives an explanation regarding an MRI examination and receives the MRI examination under a radiologist 20 in an examination room, and an assistant 30 of the subject 10 receives an explanation regarding the MRI examination in a waiting room. The medical examination system 1 includes a medical examination apparatus 40, a camera 50, a microphone 60, headphones 70, a tablet terminal 80, and an examination support apparatus 100.

The medical examination apparatus 40 is an apparatus that performs a medical examination on the subject 10. The medical examination apparatus 40 may be, for example, an MRI apparatus.

The camera 50 is an imaging device provided on a ceiling of the examination room, and images the inside of the examination room. Therefore, in a case where the subject 10 and the radiologist 20 have entered the examination room, the camera 50 can image the subject 10 and the radiologist 20. The camera 50 transmits the captured imaging data to the examination support apparatus 100 via a network (not illustrated).

The microphone 60 is a sound collecting device provided on a ceiling of the examination room, and collects the voice in the examination room. Therefore, in a case where the subject 10 and the radiologist 20 have entered the examination room, the microphone 60 can collect the voice of the subject 10 and the radiologist 20. The microphone 60 transmits the collected audio data to the examination support apparatus 100 via the network.

The headphones 70 are an example of a presentation device that presents information related to the medical examination, and are an acoustic device that presents the information related to the medical examination using voice. In general, in the MRI examination, an image is captured by causing a current to flow to a magnet in the MRI apparatus. In this case, the coil in the MRI apparatus vibrates and generates a loud sound. Therefore, the MRI apparatus is often provided with headphones in order to reduce a burden on the subject 10. In the medical examination system 1, the headphones already installed in the medical examination apparatus 40 may be used as an acoustic device that presents information related to the medical examination using voice. Note that, in the above description, a case where the headphones are used as the acoustic device is described as an example, but another device capable of outputting voice, such as a speaker (not illustrated), may be used as the acoustic device.

The tablet terminal 80 is an example of a presentation device that presents information related to the medical examination, and is a display device that presents information related to the medical examination by display. Note that the tablet terminal 80 may be a display device that presents information related to the medical examination by display, and may be an acoustic device that presents information related to the medical examination by voice.

The examination support apparatus 100 supports the medical examination by making the information related to the medical examination into a format according to a person who is a presentation destination. Note that, in FIG. 1, a case where the examination support apparatus 100 is an apparatus independent of the medical examination apparatus 40 is illustrated as an example, but the present disclosure is not limited thereto. The examination support apparatus 100 may be mounted on the medical examination apparatus 40. As a result, a function of supporting the examination and a function of performing the examination may be provided by one apparatus.

In such a medical examination system 1, in a case where a medical examination such as an MRI examination is performed, the subject 10 and the assistant 30 may be presented with explanations, precautions, and the like related to the medical examination in advance. In addition, during the examination, the subject 10 may be presented with an instruction (such as “please lie on your back” or “please take a deep breath and hold it”) related to the medical examination. In such a case, the examination support apparatus 100 according to the present embodiment makes information related to the medical examination into a format according to a person who is the presentation destination. This will be described in detail.

FIG. 2 is a diagram illustrating an example of a hardware configuration of the examination support apparatus 100 according to the present embodiment. The examination support apparatus 100 includes a processor 101, a read only memory (ROM) 102, a random access memory (RAM) 103, a storage 104, a communication interface 105, and a user interface 106. These configurations are communicably connected to each other via a bus 109.

The processor 101 executes various programs, and controls each configuration. The ROM 102 stores various programs and various kinds of data. The RAM 103 temporarily stores a program or data as a working area. The storage 104 is configured with a hard disk drive (HDD) or a solid state drive (SSD), and stores various kinds of data and various programs including an operating system.

The communication interface 105 is an interface through which the examination support apparatus 100 communicates with other apparatuses. The user interface 106 is an input and output interface through which the examination support apparatus 100 exchanges information with the user. The user interface 106 may include, for example, a touch panel, a keyboard, a microphone, and the like as an input device, and may include a monitor, a speaker, and the like as an output device.

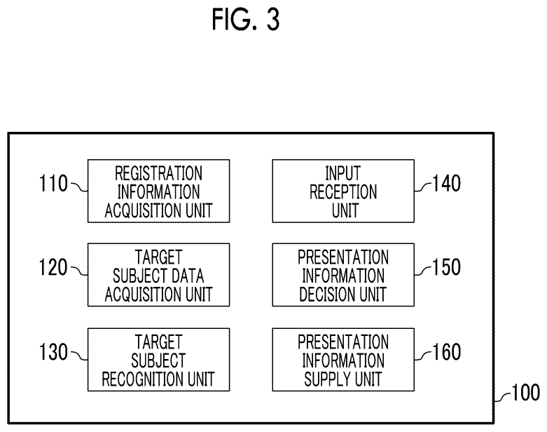

FIG. 3 is a diagram illustrating an example of a functional configuration of the examination support apparatus 100 according to the present embodiment. The examination support apparatus 100 according to the present embodiment includes a registration information acquisition unit 110, a target subject data acquisition unit 120, a target subject recognition unit 130, an input reception unit 140, a presentation information decision unit 150, and a presentation information supply unit 160. These functional configurations are realized by the processor 101 reading out the examination support program from the ROM 102 or the storage 104 and developing the examination support program in the RAM 103.

The registration information acquisition unit 110 acquires registration information registered in advance for the target subject who is the presentation destination of the information related to the medical examination.

The target subject data acquisition unit 120 acquires target subject data including at least one of imaging data obtained by imaging a target subject who is a presentation destination of the information related to the medical examination or audio data obtained by collecting the voice of the target subject.

The target subject recognition unit 130 recognizes a target subject by using the target subject data. The target subject recognition unit 130 may recognize the attributes of the target subject by using the target subject data. In addition, the target subject recognition unit 130 may recognize a situation of the target subject during the viewing of the information related to the medical examination by using the target subject data.

In a case where a plurality of target subjects having different attributes are recognized, the input reception unit 140 receives a user's input for selecting which target subject among the plurality of target subjects the presentation information is presented for.

The presentation information decision unit 150 decides the presentation information to be presented for the target subject by the presentation device that presents the information related to the medical examination, on the basis of the target subject data.

The presentation information supply unit 160 supplies the presentation information to the presentation device.

A method of supporting the medical examination by the examination support apparatus 100 including such a functional unit will be described in detail. First, a case where the information related to the medical examination is presented for the subject 10 will be described. Subsequently, a case where the information related to the medical examination is presented for the assistant 30 will be described.

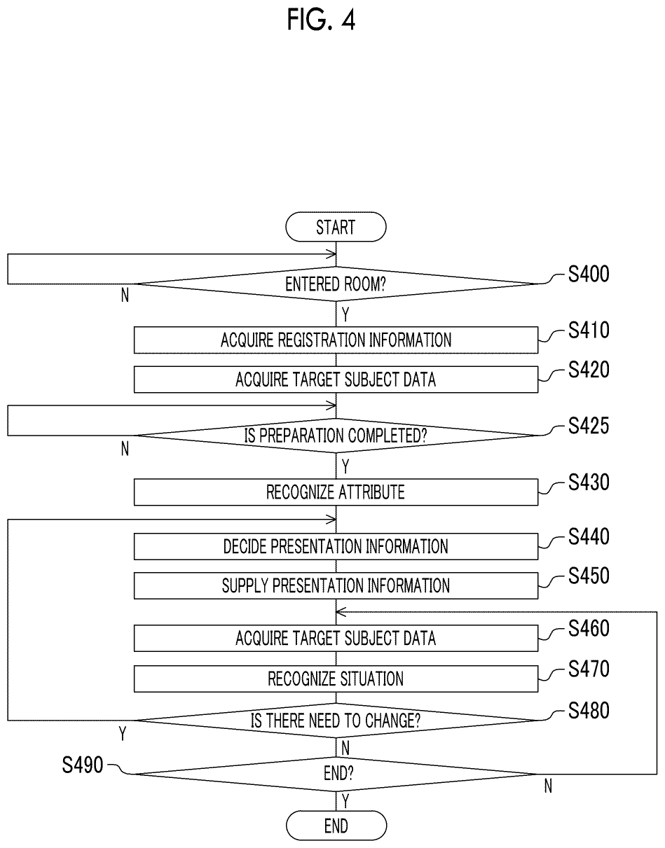

FIG. 4 is a diagram illustrating an example of a flow in a case where the information related to the medical examination is presented for the subject 10. The present flow is executed by the processor 101 reading out the examination support program from the ROM 102 or the storage 104 and developing the examination support program in the RAM 103. The same applies to the flow described below. In the present flow, the target subject who is the presentation destination of the information related to the medical examination is the subject 10 himself/herself.

In step S400, the processor 101 determines whether or not the subject 10 has entered the examination room. For example, the processor 101 may determine that the subject 10 has entered the examination room in response to the reading of a barcode printed on a wristband worn on the arm of the subject 10 by a barcode reader provided in the examination room. However, the present disclosure is not limited to this configuration. For example, the processor 101 may determine that the subject 10 has entered the examination room in response to the pressing of an entrance check button by the radiologist 20, who has checked that the subject 10 has entered the examination room. In addition, the processor 101 can determine that the subject 10 has entered the examination room on the basis of at least any of the imaging data or the audio data.

In step S410, as the registration information acquisition unit 110, the processor 101 acquires the registration information registered in advance regarding the target subject who is the presentation destination of the information related to the medical examination, here, regarding the subject 10 via the communication interface 105. For example, the processor 101 may access at least any of a hospital information system (HIS) or a radiology information system (RIS). Then, the processor 101 may acquire the registration information registered in advance regarding the subject 10 by searching for an identification number identified from the barcode read in step S400 as a search key. As an example, the processor 101 may acquire the name, height, weight, and examination information of the subject 10 as the registration information.

In step S420, as the target subject data acquisition unit 120, the processor 101 acquires the target subject data including at least one of the imaging data obtained by imaging the target subject who is the presentation destination of the information related to the medical examination, here, imaging the subject 10, or the audio data obtained by collecting the voice of the target subject, via the communication interface 105. For example, the processor 101 may acquire the imaging data obtained by imaging the subject 10 by the camera 50, as the target subject data. The imaging data acquired in this case may include, for example, data of at least any of a three-dimensional image captured by a 3D camera, an infrared image captured by an infrared camera, or a video captured by a video camera. Instead of or in addition to this, the processor 101 may acquire the audio data obtained by collecting the voice of the subject 10 by the microphone 60, as the target subject data.

However, the present disclosure is not limited to this configuration. For example, the processor 101 may acquire the imaging data captured by a camera provided on a gantry front surface of the MRI apparatus, the audio data collected by a microphone provided on the gantry front surface, the audio data collected by a microphone integrally configured with the headphones 70, or the like, as the target subject data.

In step S425, the processor 101 determines whether or not the viewing preparation of the subject 10 is completed. For example, the processor 101 may automatically recognize that the viewing preparation of the subject 10 is completed, from the target subject data acquired in step S420. However, the present disclosure is not limited to this configuration. For example, the processor 101 may determine that the viewing preparation of the subject 10 is completed, in response to the detection of the wearing of the headphones 70. In addition, for example, the processor 101 may determine that the viewing preparation of the subject 10 is completed in response to the pressing of a preparation check button by the radiologist 20, who has checked that the viewing preparation of the subject 10 is completed.

In step S430, as the target subject recognition unit 130, the processor 101 recognizes the attributes of the target subject, here, the subject 10, by using the target subject data acquired in step S420. For example, the processor 101 may recognize, as the attributes of the subject 10, the gender, the age, the language used, the hometown, the impairment information, and the like of the subject 10 from the appearance, the gesture, the wording, the voice quality, the accent, the tone, the smoothness of speech, and the like of the subject 10.

In this case, in a case where the target subject data includes the imaging data or audio data regarding a person other than the subject 10, for example, regarding the radiologist 20, the processor 101 may select or discard the target subject data on the basis of the registration information acquired in step S410. For example, it is assumed that it is known from the registration information that the subject 10 has a height of 130 cm and a weight of 25 kg. In this case, the processor 101 may exclude data other than an estimated height of 130 cm±10 cm and data other than an estimated weight of 25 kg±10 kg estimated from the imaging data, from the target subject data.

In addition, the processor 101 may determine whether the data is the data regarding the subject 10 or the data regarding the radiologist 20 on the basis of the position of the subject in the imaging data and a pointing direction in the audio data, and may select or discard the target subject data according to the determination result. Furthermore, the processor 101 may determine whether the subject 10 is speaking or the radiologist 20 is speaking on the basis of the movement of the mouth (movement of the mask in a case where the mask is worn), and may select or discard the target subject data according to the determination result.

In step S440, as the presentation information decision unit 150, the processor 101 decides the presentation information to be presented for the target subject, here, the subject 10 by the presentation device that presents the information related to the medical examination, on the basis of the target subject data. In step S440 following step S430, the processor 101 decides the presentation information on the basis of the attributes of the subject 10 recognized in step S430.

For example, it is assumed that “gender=female”, “age=less than 12 years old”, “language used=Japanese”, “hometown=Kansai”, “visual impairment=yes”, and “hearing impairment=no” are recognized as the attributes of the subject 10. In this case, the processor 101 may first select the acoustic device, for example, the headphones 70 as the presentation device that presents the information related to the medical examination, on the basis of the impairment information. Then, the processor 101 may decide the presentation information by deciding the voice output for the subject 10 by the headphones 70 to be “frequency=high”, “volume=small”, “speed=slow”, and “language=Japanese (Kansai dialect)”. This is generally based on empirical rules such as women feeling more comfortable with women's voice, children having good hearing, children being slow to understand information, Japanese speakers being able to understand Japanese, and Kansai people feeling familiar with the Kansai dialect.

As described above, in a case where the presentation device includes, for example, an acoustic device that presents information related to the medical examination by voice, such as the headphones 70, the processor 101 may decide at least one of the frequency, the volume, the speed, or the language of the voice in deciding the presentation information.

In step S450, the processor 101 supplies the presentation information decided in step S440 to the presentation device, here, the headphones 70, via the communication interface 105. As a result, the information related to the medical examination is output from the headphones 70 in a format according to the attributes of the subject 10. More specifically, the information related to the medical examination is output as voice in Japanese from the headphones 70 using a small voice of a female person and a slow Kansai dialect.

In step S460, as the target subject data acquisition unit 120, the processor 101 acquires the target subject data including at least one of the imaging data obtained by imaging the target subject, here, the subject 10 during the viewing of the information related to the medical examination or the audio data obtained by collecting the voice of the target subject, via the communication interface 105. The acquisition of the target subject data itself may be the same as that in step S420, and the description thereof will be omitted here.

In step S470, as the target subject recognition unit 130, the processor 101 recognizes a situation of the target subject, here, the subject 10 during the viewing of the information related to the medical examination by using the target subject data acquired in step S460. For example, the processor 101 may recognize the posture, the line of sight, the facial expression, the utterance, and the like of the subject 10.

In step S480, the processor 101 determines whether or not there is a need to change the presentation information, on the basis of the situation of the subject 10 recognized in step S470. For example, in a case where the processor 101 ascertains from the situation of the subject 10 that the subject 10 is dozing off, looking away, or making a facial expression or utterance indicating that the meaning of the presentation information is not understood, the processor 101 may determine that there is a need to change the presentation information. In this case, the processor 101 causes the processing to return to step S440.

In step S440 following step S480, the processor 101 decides the presentation information on the basis of the situation of the target subject, here, the subject 10, which is recognized in step S470. For example, in a case where it is ascertained that the subject 10 is dozing off, the processor 101 may decide the presentation information such that the information related to the medical examination is temporarily stopped and voice of “please wake up” is output for the subject 10 from the headphones 70. In addition, in a case where it is ascertained that the subject 10 is looking away, the processor 101 may decide the presentation information such that voice of “please concentrate on the examination information” is output for the subject 10 from the headphones 70, in addition to the information related to the medical examination. In addition, in a case where it is ascertained that the subject 10 makes a facial expression or utterance indicating that the meaning of the presented presentation information is not understood, the processor 101 may decide the presentation information such that the information related to the medical examination is output as voice from the headphones 70 more slowly or repeatedly. As a result, the presentation information according to a viewing situation of the subject 10 is output as voice from the headphones 70.

On the other hand, in step S480, in a case where it is determined that there is no need to change the presentation information, the processor 101 causes the processing to proceed to step S490. In step S490, the processor 101 determines whether or not the presentation of the information related to the medical examination is ended. In a case where it is determined that the presentation of the information related to the medical examination is not ended, the processor 101 causes the processing to return to step S460. That is, the processor 101 repeats the processing of monitoring the situation of the subject 10 and changing the presentation information as necessary until the presentation of the information related to the medical examination is ended.

For example, the examination support apparatus 100 according to the present embodiment can make the information related to the medical examination into a format according to the subject 10 in this manner. Subsequently, a case where the information regarding the medical examination is presented for the assistant 30 will be described.

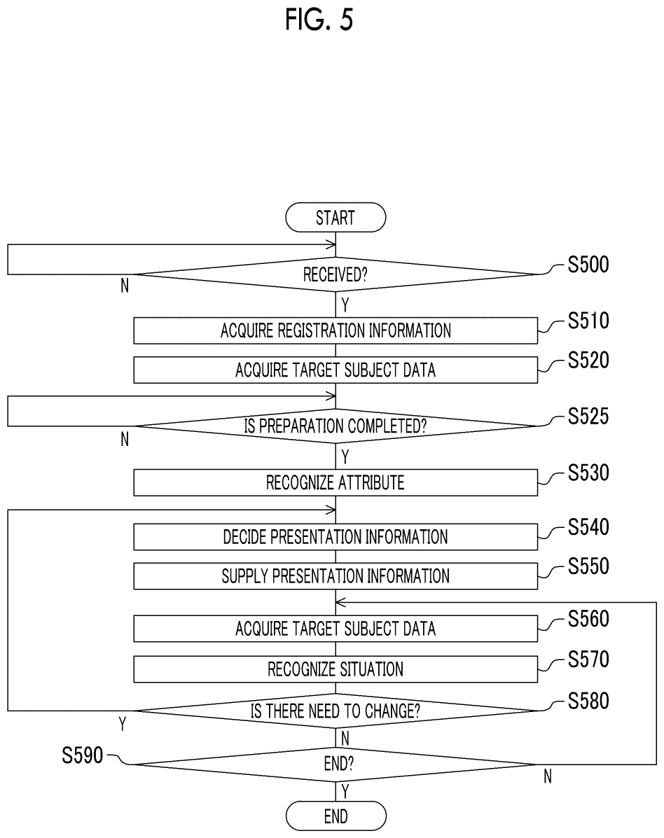

FIG. 5 is a diagram illustrating an example of a flow in a case where the information related to the medical examination is presented for the assistant 30. In the present flow, the target subject who is the presentation destination of the information related to the medical examination is not the subject 10 himself/herself, but the assistant 30.

In step S500, the processor 101 determines whether or not the target subject, here, the assistant 30 has received the tablet terminal 80. For example, the processor 101 may determine that the assistant 30 has received the tablet terminal 80 in response to the pressing of a reception check button by a nurse who has checked that the assistant 30 has received the tablet terminal 80.

In step S510, as the registration information acquisition unit 110, the processor acquires the registration information registered in advance regarding the target subject who is the presentation destination of the information related to the medical examination, here, regarding the assistant 30, via the communication interface 105. For example, the processor 101 may access at least one of the HIS or the RIS. Then, the processor 101 may search for the registration information registered in advance regarding the assistant 30 by using, as a search key, the name of the assistant 30 input by the assistant 30 via the tablet terminal 80.

However, here, it is assumed that the information regarding the assistant 30 is not registered in advance. In this case, the processor 101 may display a screen for prompting the input of the name of the subject 10 on the tablet terminal 80. Moreover, the processor 101 may acquire the examination information of the subject 10 by searching at least any of the HIS or the RIS by using the name of the subject 10 input via the tablet terminal 80 as a search key.

In step S520, as the target subject data acquisition unit 120, the processor 101 acquires the target subject data including at least one of the imaging data obtained by imaging the target subject who is the presentation destination of the information regarding the medical examination, here, imaging the assistant 30, or the audio data obtained by collecting the voice of the target subject, via the communication interface 105. For example, the processor 101 may acquire, as the target subject data, the imaging data obtained by imaging the assistant 30 with the camera built in the tablet terminal 80. Instead of or in addition to this, the processor 101 may acquire the audio data obtained by collecting the voice of the assistant 30 by the microphone built in the tablet terminal 80, as the target subject data.

However, the present disclosure is not limited to this configuration. For example, the processor 101 may acquire, as the target subject data, the imaging data captured by the camera provided on the ceiling of the waiting room or the audio data collected by the microphone provided on the ceiling of the waiting room.

In step S525, the processor 101 determines whether or not the viewing preparation of the assistant 30 is completed. For example, the processor 101 may automatically recognize that the viewing preparation of the assistant 30 is completed, from the target subject data acquired in step S520. However, the present disclosure is not limited to this configuration. For example, the processor 101 may determine that the viewing preparation of the assistant 30 is completed in response to the pressing of a viewing start button displayed on the screen of the tablet terminal 80 by the assistant 30.

In step S530, as the target subject recognition unit 130, the processor 101 recognizes the attributes of the target subject, here, the assistant 30, by using the target subject data acquired in step S520. For example, the processor 101 may recognize, as the attributes of the assistant 30, the gender, the age, the language used, the hometown, the impairment information, and the like of the assistant 30 from the appearance, the gesture, the wording, the voice quality, the accent, the tone, the smoothness of speech, and the like of the assistant 30.

In step S540, as the presentation information decision unit 150, the processor 101 decides the presentation information to be presented for the target subject, here, the assistant 30 by the presentation device that presents the information regarding the medical examination, on the basis of the target subject data. In step S540 following step S530, the processor 101 decides the presentation information on the basis of the attributes of the assistant 30 recognized in step S530.

For example, it is assumed that “gender=female”, “age=65 years old or older”, “language used=Korean”, “hometown=unknown”, “visual impairment=no”, and “hearing impairment=yes” are recognized as the attributes of the assistant 30. In this case, the processor 101 may first select the display device, for example, the tablet terminal 80 as the presentation device that presents the information related to the medical examination, on the basis of the impairment information. Then, the processor 101 may decide the presentation information by deciding the display output by the tablet terminal 80 toward the assistant 30 to be “color=black characters on yellow ground”, “brightness=bright”, “character size=large”, “speed=slow”, and “language=Korean”. This is generally based on empirical rules such as a color or brightness that is easy to see being changed as people age, elderly people having difficulty reading small letters, elderly people being slow to understand information, and Korean speakers being able to understand Korean.

As described above, in a case where the presentation device includes, for example, a display device that presents the information related to the medical examination by display, such as the tablet terminal 80, the processor 101 may decide at least one of a color, a brightness, a size, a speed, or a language of the display in deciding the presentation information.

In step S550, the processor 101 supplies the presentation information decided in step S540 to the presentation device, here, the tablet terminal 80, via the communication interface 105. As a result, the information regarding the medical examination is output from the tablet terminal 80 in a format according to the attributes of the assistant 30. More specifically, the information regarding the medical examination is output from the tablet terminal 80, as display, slowly in Korean in large black letters on the yellow background with brightness.

In step S560, as the target subject data acquisition unit 120, the processor 101 acquires the target subject data including at least one of the imaging data obtained by imaging the target subject, here, the assistant 30 during the viewing of the information related to the medical examination or the audio data obtained by collecting the voice of the target subject, via the communication interface 105. The acquisition of the target subject data itself may be the same as that in step S520, and the description thereof will be omitted here.

In step S570, as the target subject recognition unit 130, the processor 101 recognizes a situation of the target subject, here, the assistant 30 during the viewing of the information related to the medical examination by using the target subject data acquired in step S560, and the processor may recognize, for example, the posture, the line of sight, the facial expression, the utterance, and the like of the assistant 30.

In step S580, the processor 101 determines whether or not there is a need to change the presentation information, on the basis of the situation of the assistant 30 recognized in step S570. For example, in a case where the processor 101 ascertains from the situation of the assistant 30 that the assistant 30 is dozing off, looking away, or making a facial expression or utterance indicating that the meaning of the presentation information is not understood, the processor 101 may determine that there is a need to change the presentation information. In this case, the processor 101 causes the processing to return to step S540.

In step S540 following step S580, the processor 101 decides the presentation information on the basis of the situation of the target subject, here, the assistant 30, which is recognized in step S570. For example, in a case where it is ascertained that the assistant 30 is dozing off, the processor 101 may decide the presentation information such that the information related to the medical examination is temporarily stopped and a display of “please wake up” is output from the tablet terminal 80 toward the assistant 30 while vibrating the vibration. In addition, in a case where it is ascertained that the assistant 30 is looking away, the processor 101 may decide the presentation information such that a display of “please concentrate on the examination information” is output while turning on and off the flashlight, in addition to the information related to the medical examination. In addition, in a case where it is ascertained that the assistant 30 makes a facial expression or utterance indicating that the meaning of the presented presentation information is not understood, the processor 101 may decide the presentation information such that the information related to the medical examination is output, as display, from the tablet terminal 80 more slowly or repeatedly. As a result, the presentation information according to a viewing situation of the assistant 30 is output, as display, from the tablet terminal 80.

On the other hand, in step S580, in a case where it is determined that there is no need to change the presentation information, the processor 101 causes the processing to proceed to step S590. In step S590, the processor 101 determines whether or not the presentation of the information related to the medical examination is ended. In a case where it is determined that the presentation of the information related to the medical examination is not ended, the processor 101 causes the processing to return to step S560. That is, the processor 101 repeats the processing of recognizing the situation of the assistant 30 and changing the presentation information as necessary until the presentation of the information related to the medical examination is ended.

For example, the examination support apparatus 100 according to the present embodiment can make the information related to the medical examination into a format according to the assistant 30 in this manner.

In the related art, in a case where a person who is the presentation destination of the information is not registered in advance or is insufficiently registered, the information related to the medical examination may not be made into a format according to the person who is the presentation destination. In this case, the flow or the content of the medical examination is presented in a general format, but in a case where a person who is the presentation destination of the information is a child or a foreigner or in a case where the languages used by the subject 10 and the assistant 30 are different, there is a problem that it takes time to explain the flow or the content of the medical examination and it is difficult to explain the flow or the content of the medical examination correctly.

Therefore, the examination support apparatus 100 according to the present embodiment acquires the target subject data including at least one of the imaging data obtained by imaging the target subject who is the presentation destination of the information related to the medical examination or the audio data obtained by collecting the voice of the target subject, and decides the presentation information to be presented for the target subject by the presentation device on the basis of the target subject data. Accordingly, with the examination support apparatus 100 according to the present embodiment, even in a case where, for example, only the name, the height, the weight, and the examination information are registered as the information regarding the subject 10 or even in a case where the subject 10 is in a critical condition and no information regarding the subject 10 is registered, the information related to the medical examination can be made into a format according to the subject 10. In addition, with the examination support apparatus 100 according to the present embodiment, even in a case where no information regarding the assistant 30 is registered, the information related to the medical examination can be made into a format according to the assistant 30.

In addition, the examination support apparatus 100 may recognize the attributes of the target subject by using the target subject data and decide the presentation information on the basis of the attributes. Accordingly, with the examination support apparatus 100, the gender, the age, the language used, the hometown, the impairment information, and the like can be automatically recognized from the appearance, the gesture, the wording, the voice quality, the accent, the tone, the smoothness of speech, and the like of the target subject such as the subject 10 or the assistant 30, and the information related to the medical examination can be made into a delicate format according to these attributes.

In addition, the examination support apparatus 100 may recognize the viewing situation of the target subject by using the target subject data, and decide the presentation information on the basis of the viewing situation. As a result, with the examination support apparatus 100, an understanding situation of the information regarding the medical examination can be automatically inferred from the posture, the line of sight, the facial expression, the utterance, and the like of the target subject such as the subject 10 or the assistant 30, and the target subject can be alerted or the format can be changed as necessary.

In addition, in a case where the information related to the medical examination is presented by voice, the examination support apparatus 100 may decide at least one of the frequency, the volume, the speed, or the language of the voice. In addition, in a case where the information related to medical examination is presented by display, the examination support apparatus 100 may decide at least one of the color, the brightness, the size, the speed, or the language of the display. Accordingly, with the examination support apparatus 100, it is possible to decide an appropriate format from among various options in order to make the information related to the medical examination into a format according to the target subject. Therefore, with the examination support apparatus 100, for example, in a case where the target subject is elderly, the frequency of the voice is lowered because the high frequency is difficult to hear, and in a case where the target subject has a visual impairment (including a case where the target subject has an eye injury), the examination support apparatus 100 can also flexibly respond to presenting the information by voice instead of the display.

One embodiment that can be performed has been described so far as an example.

However, the embodiment described above can be modified into various forms or applied to various uses. For example, in the above description, a case where the examination support apparatus 100 always acquires the target subject data has been described as an example. However, the examination support apparatus 100 may acquire the target subject data as necessary.

For example, it is assumed that, in addition to the name, the height, the weight, and the examination information of the subject 10, the gender and the age are registered in advance as the information regarding the subject 10. In this case, the examination support apparatus 100 can decide the frequency, the volume, and the speed of the voice, the color, the brightness, the size, and the speed of the display, and the like from the gender and the age. However, since the language used by the subject 10 is not known only from the registration information, the examination support apparatus 100 cannot decide which language should be used to present the information related to the medical examination. In addition, since the impairment information of the subject 10 is not known only from the registration information, the examination support apparatus 100 cannot decide whether to present the information related to the medical examination by voice or by display.

For example, in a case where the presentation information cannot be decided on the basis of the registration information, the processor 101 may instruct the imaging device, for example, the camera 50, which images the target subject, here, the subject 10, to perform imaging or instruct the sound collecting device, for example, the microphone 60, which collects the voice of the target subject, to collect the sound.

Then, the processor 101 may decide the presentation information on the basis of the registration information and the target subject data. As described above, in a case where the registration information is insufficient for deciding the presentation information, the examination support apparatus 100 may complement the information for deciding the presentation information by acquiring the target subject data. Accordingly, with the examination support apparatus 100, since there is no need to always acquire the target subject data, it is possible to reduce the power consumption of the imaging device or the sound collecting device and to avoid the congestion of a storage capacity due to the target subject data.

In addition, in the above description, a case where the target subject is one person has been described as an example. However, a case where there are a plurality of target subjects is also considered. Examples of such a case include a case where the assistant 30 enters the examination room together with the subject 10, and a case where a plurality of assistants 30 enter the waiting room. Therefore, the processor 101 can recognize a plurality of target subjects having different attributes from the target subject data.

In this manner, in a case where a plurality of target subjects having different attributes are recognized, the processor 101 may determine which of the subject 10, the radiologist 20, and the assistant 30 each target subject corresponds to, on the basis of at least any of the imaging data or the audio data. In such determination, as described above, at least any of the rate of match with the registration information, the position of the subject, the pointing direction, the movement of the mouth, or the like may be considered. Instead of or in addition to this, the processor 101 may receive a user's input for selecting which target subject among the plurality of target subjects the presentation information is provided for, as the input reception unit 140.

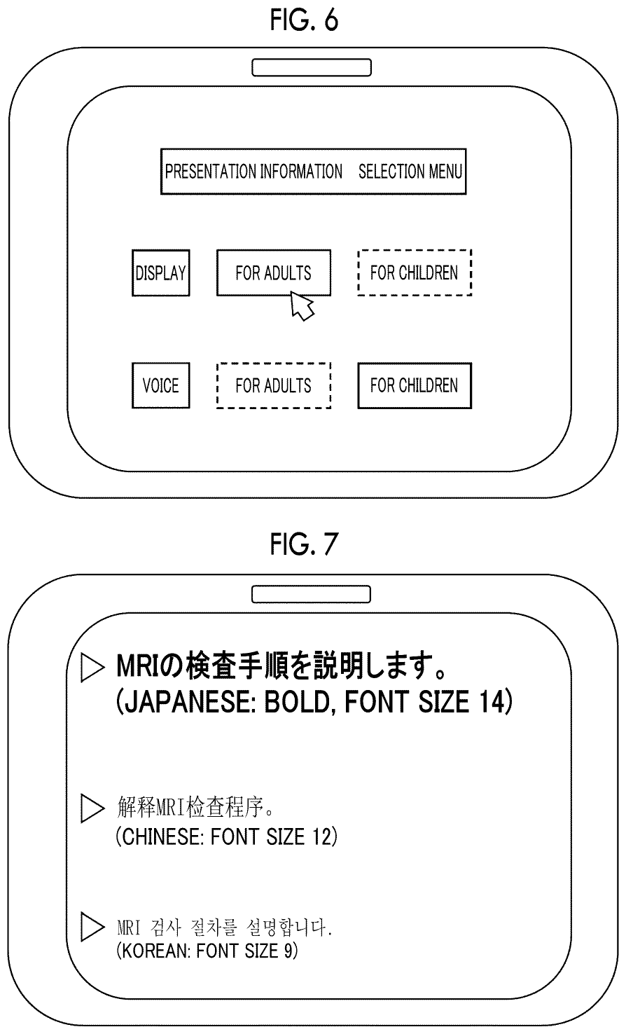

FIG. 6 is a diagram illustrating an example of the display in a case where a user's input is received. In FIG. 6, a case where the presentation device includes a first presentation device and a second presentation device that present the information related to the medical examination in different formats is illustrated as an example. Here, the first presentation device may be, for example, a display device that presents the information related to the medical examination by display, and may be a monitor of the examination support apparatus 100 as an example. The second presentation device may be, for example, an acoustic device that presents the information related to the medical examination by voice, and may be, for example, the headphones 70 as an example.

In this way, in a case where two persons of the subject 10 and the assistant 30 have entered the examination room where two presentation devices of the display device and the acoustic device are present, the processor 101 may display a screen as illustrated in FIG. 6 on, for example, the monitor of the examination support apparatus 100.

The subject 10 or the assistant 30 who sees such a screen may select whether to make the presentation information to be output, as display, from the monitor for adults, that is, for the assistant 30 or for children, that is, for the subject 10 by operating the mouse of the examination support apparatus 100. Similarly, the subject 10 or the assistant 30 may select whether to make the presentation information to be output as voice from the headphones 70 for adults or for children.

Note that, in the above description, a case where the selection is performed by a mouse operation has been described as an example, but the selection can also be performed by another operation such as voice recognition via a touch panel, a remote controller, or a microphone.

In FIG. 6, an example in which the selection of the display for adults and the voice for children is made is illustrated. In a case where such selection is made, the processor 101 may output, as display, “the information for adults is presented from this monitor” from the monitor, and output, as voice, “the information for children is presented from these headphones” from the headphones 70. As described above, the processor 101 may guide in advance as to whom information will be presented from which presentation device. Then, the processor 101 may decide the presentation information to be output, as display, for adults and the presentation information to be output, as voice, for children. For example, in this manner, in a case where the presentation device includes the first presentation device and the second presentation device that present the information related to the medical examination in different formats and a first target subject and a second target subject having different attributes are recognized, the processor 101 may decide each of first presentation information to be presented for the first target subject by the first presentation device and second presentation information to be presented for the second target subject by the second presentation device.

Note that, in the above description, a case where the display is for adults and the voice is for children has been described as an example, but the same can be applied to various cases such as a case where the display is in English and the voice is in Japanese, and a case where the display is for a hearing impaired person and the voice is for a visually impaired person.

Accordingly, with the examination support apparatus 100, even in a case where there are a plurality of target subjects, it is possible to make the information related to the medical examination into a format according to the request of the target subject. In addition, with the examination support apparatus 100, in a case where there are a plurality of presentation devices capable of presenting the information related to the medical examination in different formats, the presentation information to be presented by each of the plurality of presentation devices can be made into the format according to each of the plurality of target subjects.

Note that, in the above description, a case where the user selects which target subject among the plurality of target subjects the presentation information is to be provided for has been described as an example. However, the examination support apparatus 100 may automatically select which target subject among the plurality of target subjects the presentation information is to be provided for.

For example, a standard of “for children (less than 12 years old)> for elderly persons (65 years old or older)> for adults (12 to 64 years old)” may be determined in advance as the priority in the age. Similarly, a standard of “for female> for male” may be determined in advance as the priority in the gender. Such a standard may be determined in advance on the hospital side. Then, in a case where a plurality of target subjects having different attributes are recognized, the processor 101 may automatically select, for example, which target subject among the plurality of target subjects the information regarding the medical examination is presented for, according to the predetermined standard. As a result, with the examination support apparatus 100, even in a case where a plurality of target subjects are recognized, it is possible to eliminate the need for the intervention by the user.

In addition, in the above description, a case where the attributes of the target subject can be uniquely recognized is illustrated as an example. However, a case where the attributes of the target subject cannot be uniquely recognized is also conceivable. Examples of such a case include a case where it is possible to recognize that the target subject is an Asian person from the appearance of the target subject, but it is not possible to determine whether the target subject is Japanese, Korean, or Chinese, and as a result, it is not possible to uniquely recognize whether the language used by the target subject is Japanese, Korean, or Chinese.

As described above, in a case where the attributes of the target subject include at least the language used by the target subject and the language used cannot be uniquely recognized, the processor 101 may decide the presentation information such that the presentation device presents the information related to the medical examination for the target subject in a plurality of candidate languages.

FIG. 7 is a diagram illustrating an example of the display in a case where the presentation information is presented in a plurality of candidate languages. In a case where the language used by the target subject cannot be uniquely recognized, the processor 101 may display a screen as illustrated in FIG. 7 on, for example, the monitor of the examination support apparatus 100.

Here, a display example in a case where the recognition accuracy that the target subject is Japanese, that is, the language used is Japanese is the highest, the recognition accuracy that the target subject is Korean, that is, the language used is Korean is the lowest, and the recognition accuracy that the target subject is Chinese, that is, the language used is Chinese is between the highest and the lowest is illustrated. In such a case, the processor 101 may decide the presentation information such that, for example, the display in Japanese is performed with a font size of 14 and in bold, the display in Chinese is performed with a font size of 12, and the display in Korean is performed with a font size of 9. That is, the processor 101 may decide the degree of emphasis of the presentation according to the recognition accuracy. In addition, the processor 101 may decide the presentation information such that, for example, the display in Japanese comes first, the display in Chinese comes second, and the display in Korean comes last. That is, the processor 101 may decide the presentation order according to the recognition accuracy. For example, the processor 101 may decide the presentation information such that the presentation device presents the information related to the medical examination for the target subject in a format according to the recognition accuracy for each of the plurality of candidate languages.

Accordingly, with the examination support apparatus 100, even in a case where the language used by the target subject cannot be uniquely recognized, the presentation information is decided to be presented in a plurality of candidate languages, and therefore, it is possible to increase a possibility that the target subject can understand the information related to the medical examination in any language. In addition, with the examination support apparatus 100, since the presentation information is decided according to the recognition accuracy for each of the plurality of candidate languages, it is possible to increase the probability that the information related to the medical examination is in a format according to the target subject. In the embodiment described above, the processor refers to a processor in a broad sense, and examples of the processor include a general-purpose processor (for example, a central processing unit (CPU) or the like), and a dedicated processor (for example, a graphics processing unit (GPU), an application specific integrated circuit (ASIC), a field programmable gate array (FPGA), a programmable logic device, or the like).

In addition, the operation of the processor in the embodiment described above may be performed not only by one processor but also by cooperation of a plurality of processors provided at physically separated positions. In addition, the order of the operations of the processor is not limited to only the order described in each embodiment, and may be changed as appropriate.

In addition, as an example, it has been described in which the examination support apparatus 100 according to the embodiment described above is configured with a single apparatus, but the examination support apparatus 100 may be configured with a plurality of apparatuses.

In addition, the processing performed by the examination support apparatus 100 according to the embodiment described above may be processing performed by software, processing performed by hardware, or processing in combination thereof. In addition, the processing performed in each unit of the examination support apparatus 100 may be stored in a storage medium as a program and distributed.

In addition, the present disclosure is not limited to the above-described aspects, and various modifications can be made within the scope not departing from the gist of the present disclosure.

The following supplementary notes are further disclosed with respect to the embodiment described above.

### Supplementary Note 1

An examination support apparatus comprising:
a processor,
wherein the processor
   acquires target subject data including at least one of imaging data obtained by imaging a target subject who is a presentation destination of information related to a medical examination or audio data obtained by collecting voice of the target subject, and
   decides presentation information to be presented for the target subject by a presentation device that presents the information related to the medical examination, on the basis of the target subject data.

### Supplementary Note 2

The examination support apparatus according to Supplementary Note 1,
wherein the processor
   recognizes attributes of the target subject by using the target subject data, and
   decides the presentation information on the basis of the attributes of the target subject.

### Supplementary Note 3

The examination support apparatus according to Supplementary Note 2,
wherein in a case where a plurality of target subjects having different attributes are recognized, the processor receives a user's input for selecting which target subject among the plurality of target subjects the presentation information is presented for.

### Supplementary Note 4

The examination support apparatus according to Supplementary Note 2 or 3,
wherein in a case where a plurality of target subjects having different attributes are recognized, the processor automatically selects which target subject among the plurality of target subjects the presentation information is presented for, according to a standard determined in advance.

### Supplementary Note 5

The examination support apparatus according to any one of Supplementary Notes 2 to 4,
wherein in a case where the presentation device includes a first presentation device and a second presentation device that present the information related to the medical examination in different formats and a first target subject and a second target subject having different attributes are recognized, the processor decides each of first presentation information to be presented for the first target subject by the first presentation device and second presentation information to be presented for the second target subject by the second presentation device.

### Supplementary Note 6

The examination support apparatus according to any one of Supplementary Notes 2 to 5,
wherein in a case where the attributes of the target subject include at least a language used by the target subject and the language used cannot be uniquely recognized, the processor decides the presentation information such that the presentation device presents the information related to the medical examination for the target subject in a plurality of candidate languages.

### Supplementary Note 7

The examination support apparatus according to any one of Supplementary Notes 2 to 6,
wherein the processor decides the presentation information such that the presentation device presents the information related to the medical examination for the target subject in a format according to a recognition accuracy for each of the plurality of candidate languages.

### Supplementary Note 8

The examination support apparatus according to any one of Supplementary Notes 1 to 7,
wherein the processor
   recognizes a situation of the target subject during viewing of the information related to the medical examination by using the target subject data, and
   decides the presentation information on the basis of the situation of the target subject.

### Supplementary Note 9

The examination support apparatus according to any one of Supplementary Notes 1 to 8,
wherein the processor
   acquires registration information registered in advance for the target subject, and
   decides the presentation information on the basis of the registration information and the target subject data.

### Supplementary Note 10

The examination support apparatus according to Supplementary Note 9,
wherein in a case where the presentation information cannot be decided on the basis of the registration information, the processor instructs an imaging device, which images the target subject, to perform imaging or instructs a sound collecting device, which collects voice of the target subject, to collect sound.

### Supplementary Note 11

The examination support apparatus according to any one of Supplementary Notes 1 to 10,
wherein in a case where the presentation device includes an acoustic device that presents the information related to the medical examination by voice, the processor decides at least any of a frequency, a volume, a speed, or a language of the voice in deciding the presentation information.

### Supplementary Note 12

The examination support apparatus according to any one of Supplementary Notes 1 to 11,
wherein in a case where the presentation device includes a display device that presents the information related to the medical examination by display, the processor decides at least any of a color, a brightness, a size, a speed, or a language of the display in deciding the presentation information.

### Supplementary Note 13

A medical examination apparatus comprising:
the examination support apparatus according to any one of Supplementary Notes 1 to 12.

### Supplementary Note 14

An examination support method comprising:
via a computer,
acquiring target subject data including at least one of imaging data obtained by imaging a target subject who is a presentation destination of information related to a medical examination or audio data obtained by collecting voice of the target subject; and
deciding presentation information to be presented for the target subject by a presentation device that presents the information related to the medical examination, on the basis of the target subject data.

### Supplementary Note 15

An examination support program causing a computer to execute:
acquiring target subject data including at least one of imaging data obtained by imaging a target subject who is a presentation destination of information related to a medical examination or audio data obtained by collecting voice of the target subject; and
deciding presentation information to be presented for the target subject by a presentation device that presents the information related to the medical examination, on the basis of the target subject data.

### EXPLANATION OF REFERENCES

What is claimed is:

1. An examination support apparatus comprising:
a processor,
wherein the processor
acquires target subject data including at least one of imaging data obtained by imaging a target subject who is a presentation destination of information related to a medical examination or audio data obtained by collecting voice of the target subject, and
decides presentation information to be presented for the target subject by a presentation device that presents the information related to the medical examination, on the basis of the target subject data.

2. The examination support apparatus according to claim 1,
wherein the processor
recognizes attributes of the target subject by using the target subject data, and
decides the presentation information on the basis of the attributes of the target subject.

3. The examination support apparatus according to claim 2,
wherein in a case where a plurality of target subjects having different attributes are recognized, the processor receives a user's input for selecting which target subject among the plurality of target subjects the presentation information is presented for.

4. The examination support apparatus according to claim 2,
wherein in a case where a plurality of target subjects having different attributes are recognized, the processor automatically selects which target subject among the plurality of target subjects the presentation information is presented for, according to a standard determined in advance.

5. The examination support apparatus according to claim 2,
wherein in a case where the presentation device includes a first presentation device and a second presentation device that present the information related to the medical examination in different formats and a first target subject and a second target subject having different attributes are recognized, the processor decides each of first presentation information to be presented for the first target subject by the first presentation device and second presentation information to be presented for the second target subject by the second presentation device.

6. The examination support apparatus according to claim 2,
wherein in a case where the attributes of the target subject include at least a language used by the target subject and the language used cannot be uniquely recognized, the processor decides the presentation information such that the presentation device presents the information related to the medical examination for the target subject in a plurality of candidate languages.

7. The examination support apparatus according to claim 6,
wherein the processor decides the presentation information such that the presentation device presents the information related to the medical examination for the target subject in a format according to a recognition accuracy for each of the plurality of candidate languages.

8. The examination support apparatus according to claim 1,
wherein the processor
recognizes a situation of the target subject during viewing of the information related to the medical examination by using the target subject data, and
decides the presentation information on the basis of the situation of the target subject.

9. The examination support apparatus according to claim 1,
wherein the processor
acquires registration information registered in advance for the target subject, and
decides the presentation information on the basis of the registration information and the target subject data.

10. The examination support apparatus according to claim 9,
wherein in a case where the presentation information cannot be decided on the basis of the registration information, the processor instructs an imaging device, which images the target subject, to perform imaging or instructs a sound collecting device, which collects voice of the target subject, to collect sound.

11. The examination support apparatus according to claim 1,
wherein in a case where the presentation device includes an acoustic device that presents the information related to the medical examination by voice, the processor decides at least any of a frequency, a volume, a speed, or a language of the voice in deciding the presentation information.

12. The examination support apparatus according to claim 1,
wherein in a case where the presentation device includes a display device that presents the information related to the medical examination by display, the processor decides at least any of a color, a brightness, a size, a speed, or a language of the display in deciding the presentation information.

13. A medical examination apparatus comprising:
the examination support apparatus according to claim 1.

14. An examination support method comprising:
via a computer,
acquiring target subject data including at least one of imaging data obtained by imaging a target subject who is a presentation destination of information related to a medical examination or audio data obtained by collecting voice of the target subject; and
deciding presentation information to be presented for the target subject by a presentation device that presents the information related to the medical examination, on the basis of the target subject data.

15. A non-transitory computer-readable storage medium storing an examination support program causing a computer to execute:
acquiring target subject data including at least one of imaging data obtained by imaging a target subject who is a presentation destination of information related to a medical examination or audio data obtained by collecting voice of the target subject; and
deciding presentation information to be presented for the target subject by a presentation device that presents the information related to the medical examination, on the basis of the target subject data.
